(19) United States
(12) Patent Application Publication
(10) Pub. No.: US 2024/0358501 A1
(43) Pub. Date: Oct. 31, 2024

(54) PROSTHETIC CARDIAC VALVE DELIVERY DEVICES, SYSTEMS, AND METHODS

(71) Applicant: Shifamed Holdings, LLC, Campbell, CA (US)

(72) Inventors: Connor Gordon MULCAHY, San Francisco, CA (US); Alice YANG, Campbell, CA (US); Savina BRACHTAUSER BALCELLS, Los Gatos, CA (US); Ali SALAHIEH, Saratoga, CA (US); Jonathan Hwan OAKDEN, San Jose, CA (US)

(21) Appl. No.: 18/573,816

(22) PCT Filed: Jun. 22, 2022
(86) PCT No.: PCT/US2022/034557

§ 371 Date: Dec. 22, 2023

Related U.S. Application Data
(60) Provisional application No. 63/213,656, filed on Jun. 22, 2021.

(51) Int. Cl. A61F 2/24

(52) U.S. Cl. CPC A61F 2/2418; A61F 2230/0023

(57) ABSTRACT

A device and system for use with medical devices, such as catheter devices or systems. The device or system comprises an anchor for securing to tissue. In some examples, the device or system is used in treating a diseased native valve in a patient. The anchor may be part of a delivery device to implant a prosthetic valve. Subsequent to delivery, the components of the delivery device are actuated to secure the prosthetic valve within the diseased valve.

### CROSS-REFERENCE

This application claims the benefit of priority of U.S. Provisional Application No. 63/213,656, filed Jun. 22, 2021, herein incorporated by reference in its entirety. This application may be related to U.S. patent application Ser. No. 16/546,901, filed on Aug. 21, 2019, titled “PROSTHETIC CARDIAC VALVE DEVICES, SYSTEMS, AND METHODS,” U.S. patent application Ser. No. 16/594,946, filed on Oct. 7, 2019, titled “PROSTHETIC CARDIAC VALVE DEVICES, SYSTEMS, AND METHODS,” International Patent Application No. PCT/US2019/057082, filed on Oct. 18, 2019, titled “ADJUSTABLE MEDICAL DEVICE,” U.S. patent application Ser. No. 16/723,537, filed on Dec. 20, 2020, titled “PROSTHETIC CARDIAC VALVE DEVICES, SYSTEMS, AND METHODS,” U.S. patent application Ser. No. 16/824,576, filed on Mar. 19, 2020, titled “PROSTHETIC CARDIAC VALVE DEVICES, SYSTEMS, AND METHODS,” and International Patent Application No. PCT/US2020/027744, filed on Apr. 10, 2020, titled “MINIMAL FRAME PROSTHETIC CARDIAC VALVE DELIVERY DEVICES, SYSTEMS, AND METHODS,” the entireties of which are incorporated by reference in their entireties.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BACKGROUND

Blood flow between heart chambers is regulated by native valves—the mitral valve, the aortic valve, the pulmonary valve, and the tricuspid valve. Each of these valves is a passive one-way valve that opens and closes in response to differential pressures. Patients with valvular disease have abnormal anatomy and/or function of at least one valve. For example, a valve may suffer from insufficiency, also referred to as regurgitation, when the valve does not fully close, thereby allowing blood to flow retrograde. Valve stenosis can cause a valve to fail to open properly. Other diseases may also lead to dysfunction of the valves.

The mitral valve, for example, sits between the left atrium and the left ventricle and, when functioning properly, allows blood to flow from the left atrium to the left ventricle while preventing backflow or regurgitation in the reverse direction. Native valve leaflets of a diseased mitral valve, however, do not fully prolapse, causing the patient to experience regurgitation.

While medications may be used to treat diseased native valves, the defective valve often needs to be repaired or replaced at some point during the patient's lifetime. Existing prosthetic valves and surgical repair and/or replacement procedures may have increased risks, limited lifespans, and/or are highly invasive. Some less invasive transcatheter options are available, but most are not ideal. A major limitation of existing transcatheter mitral valve devices, for example, is that the mitral valve devices are too large in diameter to be delivered transeptally, requiring transapical access instead. Furthermore, existing mitral valve replacement devices are not optimized with respect to strength-weight ratio and often take up too much space within the valve chambers, resulting in obstruction of outflow from the ventricle into the aorta and/or thrombosis.

Thus, a new valve device that overcomes some or all of these deficiencies is desired.

### SUMMARY OF THE DISCLOSURE

A device for treating a diseased native valve in a patient is provided, the device comprising a frame structure comprising an inflow portion, and an outflow portion comprising a plurality of protruding sections configured such that a cross-section of the outflow portion forms a convex triangular shape, and a valve segment positioned radially within the frame structure, the valve segment comprising a plurality of leaflets attached to the frame structure.

In some embodiments, each of the leaflets is attached to the frame at a central attachment point along a respective side of the convex triangular cross-section.

In other embodiment, the leaflets are unsupported by the frame except at the central attachment points.

In some implementations, there is a gap between an interior diameter of the outflow portion at the protruding sections and an outflow edge of the valve segment.

In some examples, the inflow portion is substantially cylindrical.

In other embodiments, the inflow portion is flared radially outwards.

In one example, a cross-section of the inflow portion is substantially circular.

In some examples, the outflow portion has a curvature at or above zero along an entire axial length thereof.

In one embodiment, a nominal inner diameter of the outflow portion is approximately equal to an inner diameter of the inflow portion.

In some embodiments, the frame structure is configured to self-expand from a collapsed configuration to an expanded configuration, further wherein the outflow portion comprises the plurality of protruding sections when the frame structure is in the expanded configuration.

In one example, the collapsed configuration is substantially cylindrical.

In another example, the device is configured to be placed inside a stentless surgical valve.

In some embodiments, the device further includes a spiral anchor configured to be placed around the frame structure.

In another embodiment, the convex triangular shape comprises a triangle wherein an angle along an interior surface of the triangular shape is 180 degrees or less along an entire inner perimeter.

In some embodiments, the convex triangular shape has rounded corners. In oth embodiments, the convex triangular shape is an equilateral triangle.

In some embodiments, the device includes a central portion between the inflow portion and the outflow portion.

In some examples, the convex triangular shape comprises three sides, wherein a midpoint of each of the three sides is substantially radially aligned with both the central portion and with a nominal inner circumference of the frame structure.

In other examples, the plurality of protruding sections protrude outwards relative to the central portion.

A device for treating a diseased native valve in a patient is provided, the device comprising a frame structure comprising a flared inflow portion, a plurality of paddles extending from the flared inflow portion, the plurality of paddles being configured to enable grasping and/or manipulation of the frame structure during deployment, a central portion, an outflow portion that protrudes outwards relative to the central portion at a plurality of protruding sections configured such that a cross-section of the outflow portion forms a convex triangular shape, and a valve segment positioned radially within the frame structure, the valve segment comprising a plurality of leaflets attached to the frame structure.

In some embodiments, at least a portion of the plurality of paddles is non-foreshortening during deployment.

In other embodiments, a tip of the plurality of paddles is straight.

In some examples, the plurality of paddles includes at least two inflection points.

A valve replacement system is provided, comprising a frame structure comprising an inflow portion, and an outflow portion comprising a plurality of protruding sections configured such that a cross-section of the outflow portion forms a convex triangular shape, and a valve segment positioned radially within the frame structure, the valve segment comprising a plurality of leaflets attached to the frame structure, a loading tool including a central opening with an input end having a cross-section similar to the convex triangular shape of the outflow portion of the frame structure and an output end having a generally circular cross-section, and a delivery device, wherein the frame structure is configured to be passed through the loading too to facilitate sheathing of the frame structure into the delivery device.

In some embodiments, the central opening decreases in diameter from the input end to the output end.

A method of loading a valve replacement device into a delivery device is provided, comprising inserting a frame structure of the valve replacement device into a loading tool in an expanded configuration, wherein a convex triangular cross-section of the frame structure abuts a convex triangular shape of an input end of the loading tool, passing the frame structure through a central opening of the loading tool towards an output end of the loading tool, wherein the output end comprises a circular cross-section and the central opening decreases in diameter from the input end to the output end, and sheathing the frame structure into the delivery device in a collapsed configuration.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the present disclosure are utilized, and the accompanying drawings of which:

FIG. 1 shows a perspective view of an implantable valve prosthesis, in accordance with embodiments.

FIG. 2 shows a side view of the implantable valve prosthesis of FIG. 1 crimped, in accordance with embodiments.

FIG. 3 shows a side view of the implantable valve prosthesis device of FIG. 1 connected to an anchor, in accordance with embodiments.

FIG. 4 shows a perspective view of an implantable valve prosthesis, in accordance with embodiments.

FIG. 5 shows a perspective view of an implantable valve prosthesis, in accordance with embodiments.

FIG. 6 shows a perspective view of an implantable valve prosthesis, in accordance with embodiments.

FIG. 7A shows a side view of an implantable valve prosthesis with the valve segment extending proximal of the strut frame, in accordance with embodiments.

FIG. 7B shows a bottom view (i.e., from the outflow end) of the implantable valve prosthesis of FIG. 7A.

FIG. 8A shows a portion of a valve prosthesis, in accordance with embodiments.

FIG. 8B shows a bottom view of the valve prosthesis of FIG. 8A.

FIG. 8C shows a detailed side view of the valve prosthesis of FIG. 8A.

FIG. 9A shows a side view of a valve prosthesis, in accordance with embodiments.

FIG. 9B shows a bottom view of the valve prosthesis of FIG. 9A.

FIG. 10 shows a side view of a valve prosthesis, in accordance with embodiments.

FIGS. 11A-11C show a valve prosthesis with a convex triangular outflow end.

FIGS. 12A-12D show another valve frame structure with a convex triangular outflow end.

FIGS. 12E-12G show a prosthesis including the valve frame structure of FIGS. 12A-12D.

FIG. 13A shows a prosthesis similar to FIGS. 12E-12G with the leaflets closed.

FIG. 13B shows the prosthesis of FIG. 13B with the leaflets opened.

FIG. 14A shows an exemplary pin for connecting leaflets to a frame structure.

FIG. 14B shows an interior view of a frame structure with the pin of FIG. 14A viewed from the outflow end.

FIG. 14C shows another interior view of a frame structure with the pin of FIG. 14A viewed from the outflow end.

FIG. 14D shows an exterior view of a frame structure with the pin of FIG. 14A viewed from the outflow end.

FIG. 15 shows a graph of curvature vs angle around the circumference of a convex triangular outflow end of a prosthesis.

FIGS. 16A-16D show a valve frame structure with a convex triangular outflow end positioned within a surgical valve.

FIGS. 17A-17C show an exemplary loading tool for a valve frame structure having a convex triangular outflow end.

FIGS. 18A-18F show an exemplary method of loading a valve frame structure into the loading tool of FIGS. 17A-17C.

### DETAILED DESCRIPTION

In the following detailed description, reference is made to the accompanying figures, which form a part hereof In the figures, similar symbols typically identify similar components, unless context dictates otherwise. The illustrative embodiments described in the detailed description, figures, and claims are not meant to be limiting. Other embodiments may be utilized, and other changes may be made, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Although certain embodiments and examples are disclosed below, inventive subject matter extends beyond the specifically disclosed embodiments to other alternative embodiments and/or uses, and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments, however, the order of description should not be construed to imply that these operations are order dependent. Additionally, the structures, systems, and/or devices described herein may be embodied as integrated components or as separate components.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

The present disclosure is described in relation to systems, devices, or methods for treatment or replacement of a diseased native valve of the heart, for example a mitral valve. However, one of skill in the art will appreciate that this is not intended to be limiting and the devices and methods disclosed herein may be used in other anatomical areas and in other surgical procedures.

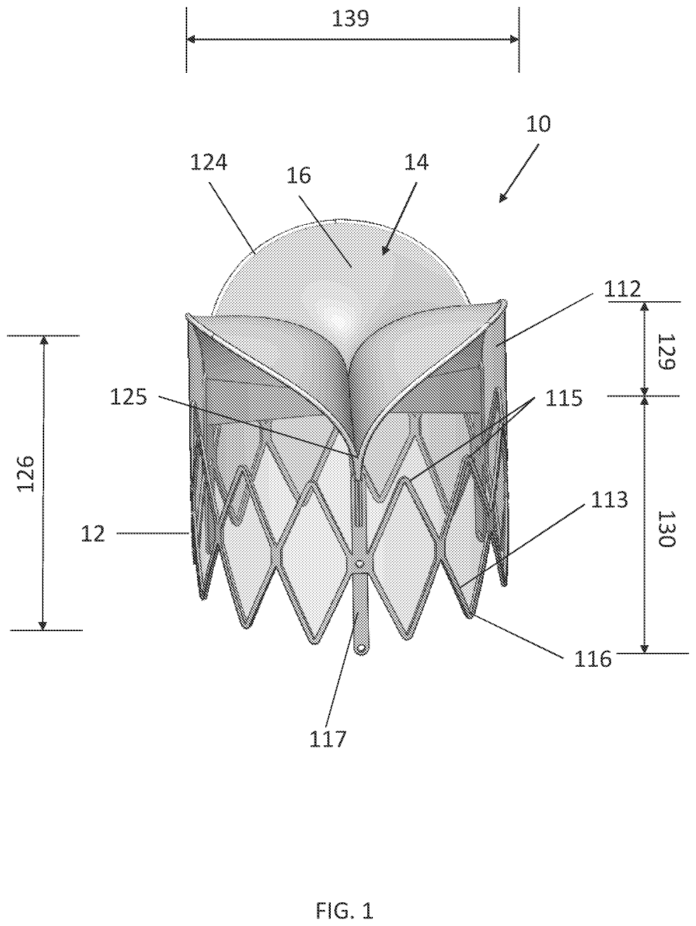

FIG. 1 shows a valve prosthesis 10 (e.g., an implantable valve prosthesis). The exemplary valve prosthesis 10 can include a frame structure 12 and a valve segment 14 positioned therein. Valve segment 14 can comprise a plurality of valve leaflets 16. In an expanded configuration, valve segment 14 can function as a fluidic valve in place of a native valve tissue (e.g., a heart valve, such as the mitral valve). The frame structure 12 can provide circumferential strength and/or longitudinal strength to valve prosthesis device 10.

One or more portions of valve prosthesis 10 can be shaped or configured to aid in securing valve prosthesis 10 at a location (e.g., in the orifice of a native heart valve). Described herein, for example, are various embodiments of anchors (e.g., spiral anchors 15) and frame flared portions that can aid in establishing or maintaining the valve prosthesis 10 at a location. In some embodiments, the valve prosthesis 10 can comprise one or more hook, barb, or scallop-shaped anchor to aid in deployment and/or positioning of valve prosthesis 10 at a location. In some cases, one or more hooks, barbs, or scallop-shaped anchor may be coupled to a portion of frame structure 12 (e.g., at a commissural post 117, a strut 113, a proximal arch 115, or a distal arch 116). For example, the frame structure 12 may comprise one or more hooks or barbs (e.g., connected to a strut 113), which can contact a tissue of a native heart valve or a tissue surrounding a native heart valve to prevent valve prosthesis 10 from moving or becoming dislodged from a location at which it has been placed or deployed.

FIG. 1 shows the valve prosthesis 10 in an expanded configuration. The valve prosthesis 10 can be deployed in an expanded configuration according to the methods described herein. For example, valve prosthesis 10 can be deployed into an expanded configuration in a method of replacing or repairing. In the expanded configuration, valve prosthesis 10 can be positioned and/or anchored at a target region of a subject (e.g., an organ or tissue of an animal such as a dog, cat, horse, or human). For example, valve prosthesis 10 can be positioned in the expanded configuration in the orifice of a heart valve, such as the mitral valve or tricuspid valve (e.g., to function as a temporary or permanent replacement for an existing mitral valve or tricuspid valve of the heart).

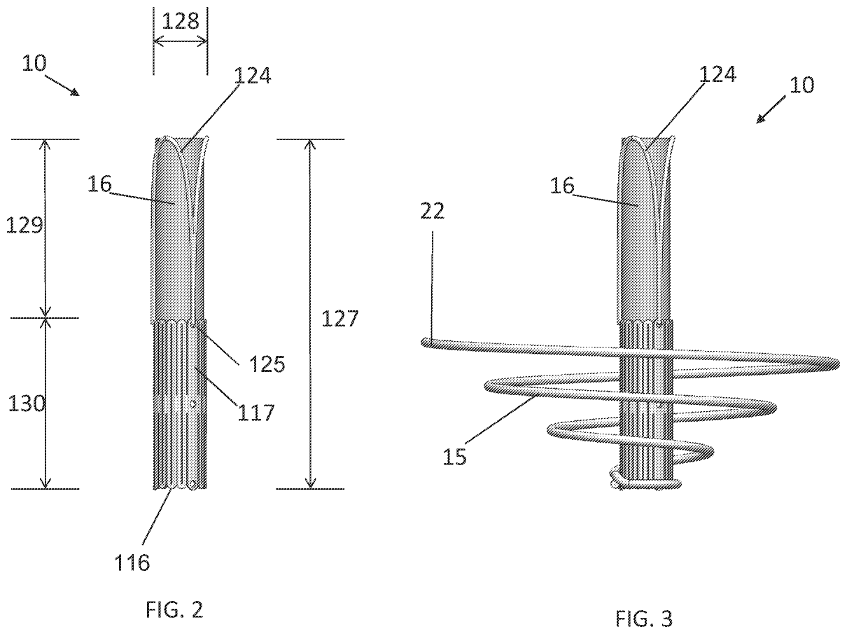

FIG. 2 shows the valve prosthesis 10 in an unexpanded (or collapsed or crimped) configuration. In some cases, the valve prosthesis 10 can be delivered to a target region (e.g., a region of a heart comprising a native valve) in the unexpanded configuration. In some cases, the valve prosthesis 10 in the unexpanded configuration can allow the valve prosthesis 10 to be delivered via minimally invasive means (e.g., via a delivery device, as described herein).

Referring to FIG. 2, the longitudinal length 127 of the collapsed valve prosthesis 10 can be minimized, which can be advantageous for delivery of the valve prosthesis 10. For example, minimizing the overall longitudinal length 127 of the collapsed valve prosthesis 10 can allow improved maneuverability within a delivery device while maintaining structural strength of the device. In some cases, minimizing the overall longitudinal length 127 of the collapsed valve prosthesis 10 can allow insertion of valve prosthesis 10 through an access path that would be challenging for a longer device to traverse (e.g., an access path comprising tortuous passages or passages with sharp turns). In some cases, the valve prosthesis 10 in the unexpanded configuration has an overall longitudinal length 127 of from 1 mm to 50 mm, from 1 mm to 45 mm, from 1 mm to 40 mm, from 1 mm to 35 mm, from 1 mm to 30 mm, from 1 mm to 25 mm, from 1 mm to 20 mm, from 1 mm to 10 mm, from 10 mm to 45 mm, from 20 mm to 45 mm, from 20 mm to 30 mm, from 25 mm to 35 mm, or from 27.5 mm to 32.5 mm. In some cases, the prosthetic delivery device 10 in the expanded In some cases, the prosthetic delivery device 10 in the expanded configuration can have an overall longitudinal length of from 1 mm to 45 mm, from 10 mm to 45 mm, from 15 mm to 45 mm, from 15 mm to 35 mm, from 16 mm to 34 mm, from 17 mm to 33 mm, from 18 mm to 32 mm, from 19 mm to 31 mm, from 20 mm to 30 mm, from 25 mm to 35 mm, or from 27.5 mm to 32.5 mm. In some embodiments, the valve prosthesis 10 can foreshorten as it expands such that the length 126 in the expanded configuration is less than the length 127 in the collapsed configuration.

Further, the diameter 128 of the collapsed valve prosthesis 10 can be minimized, which can likewise be advantageous for delivery of the valve prosthesis 10. For example, a collapsed valve prosthesis 10 with a smaller diameter 128 can fit inside of a delivery device with a smaller diameter, allowing for less invasive delivery and for improved maneuvering capability inside of a subject's body. Reducing the diameter 128 of the collapsed valve prosthesis 10 (e.g., for use in treatment or replacement of a mitral valve, a tricuspid valve, an aortic valve, or a pulmonic valve) can further allow for easier delivery of the valve prosthesis 10 to a target region of a subject, faster recovery of a subject receiving valve prosthesis 10, and/or improved clinical outcomes for a subject receiving valve prosthesis 10 (e.g., improved subject survival, improved ejection fraction, improved cardiac output, decreased valvular regurgitation, and/or decreased edema). In some cases, reducing the diameter 128 of the collapsed valve prosthesis 10 can make transeptal access and delivery possible in addition to transapical access. In some cases, the diameter 128 of the collapsed valve prosthesis 10 or portion thereof (e.g., frame structure 12) can be from 0.01 mm to 20 mm, 0.01 mm to 15 mm, 0.01 mm to 10 mm, from 0.01 mm to 9 mm, from 0.01 mm to 8 mm, from 0.01 mm to 7 mm, from 0.01 mm to 6 mm, from 0.01 mm to 5 mm, from 0.01 mm to 4 mm, from 0.01 mm to 3 mm, from 0.01 mm to 2 mm, from 0.01 mm to 1 mm, from 1 mm to 15 mm, from 2 mm to 14 mm, from 3 mm to 13 mm, from 4 mm to 12 mm, from 5 mm to 10 mm, from 6 mm to 10 mm, from 7 mm to 10 mm, from 8 mm to 10 mm, from 9 mm to 10 mm, from 10 mm to 15 mm, no more than 20 mm, no more than 15 mm, no more than 10 mm, no more than 9 mm, no more than 8 mm, no more than 7 mm, no more than 6 mm, or no more than 5 mm.

The diameter 139 of frame structure 12 in an expanded configuration (see FIG. 1) can be larger than the diameter 128 of frame structure 12 in an unexpanded configuration (see FIG. 2). In some cases, frame structure 12 or a portion thereof (e.g., annular central portion 158 of frame structure 12) can have an expanded diameter 139 of from 10 mm to 50 mm, from 20 mm to 40 mm, from 25 mm to 35 mm, from 27 mm to 33 mm, no more than 50 mm, no more than 40 mm, no more than 35 mm, no more than 33 mm, no more than 30 mm, no more than 25 mm, no more than 20 mm, or no more than 15 mm when frame structure 12 is in an expanded configuration.

In some cases, the diameter 128 or 139 refers to a largest cross-sectional width of valve prosthesis 10 or a portion thereof, e.g., as measured in a plane perpendicular to a longitudinal axis of the valve prosthesis 10 at a longitudinal location. In some situations, the valve prosthesis 10 has a polygonal cross-section. In some cases, the diameter 128, 139 can refer to the largest distance from a first side of a polygonal cross-section of the valve prosthesis 10 to a second side of the polygonal cross-section of the valve prosthesis 10.

In some cases, the valve prosthesis 10 or a portion thereof can be sized or shaped to be positioned at a certain location or target region. For example, the frame structure 12 can be sized to be positioned in a valve, such as the mitral valve (e.g., by designing a dimension of frame structure to fit a valve, such as the mitral valve, when in an expanded configuration).

As shown in FIGS. 1-2, the valve prosthesis 10 can include a first portion 129 comprising only the valve segment 14 and/or minimal valve supports 124 and a second portion 130 comprising the frame structure 12 and the valve segment 14. In some embodiments, the valve segment 14 can be entirely unsupported or mostly unsupported in the first portion 129 while the valve segment 14 can be completely supported in the second portion 130 (e.g., by the frame structure 12). For example, minimal valve supports 124 can extend from the frame structure 12 to support the valve segment 14 in the first portion 129. The minimal valve supports 124 can, for example, support only the inflow edges of the valve segment 14 in the first portion 129 while leaving the rest of the valve segment 14 unsupported in the first portion 129. The first portion 129 of the valve prosthesis 10 can be coupled to or continuous with the second portion 130. For example, the frame structure 12 can be coupled to the minimal valve supports 124 at a joint 125 (e.g., with a fastener or crimp) or can be continuous with the minimal valve supports 124 (e.g., via fusion, welding, or formation by a continuous piece of material). Further, the valve segment 14 can be coupled to the minimal valve supports 124 in the first portion 129 and to the frame structure 12 in the section portion 130. When, for example, the valve prosthesis 10 is deployed in an orifice of the native mitral valve, the valve prosthesis 10 can be oriented such that the first portion 129 is positioned closer to the atrium than the second portion 130, and the second portion 130 can be positioned closer to the ventricle of the heart than the first portion 129.

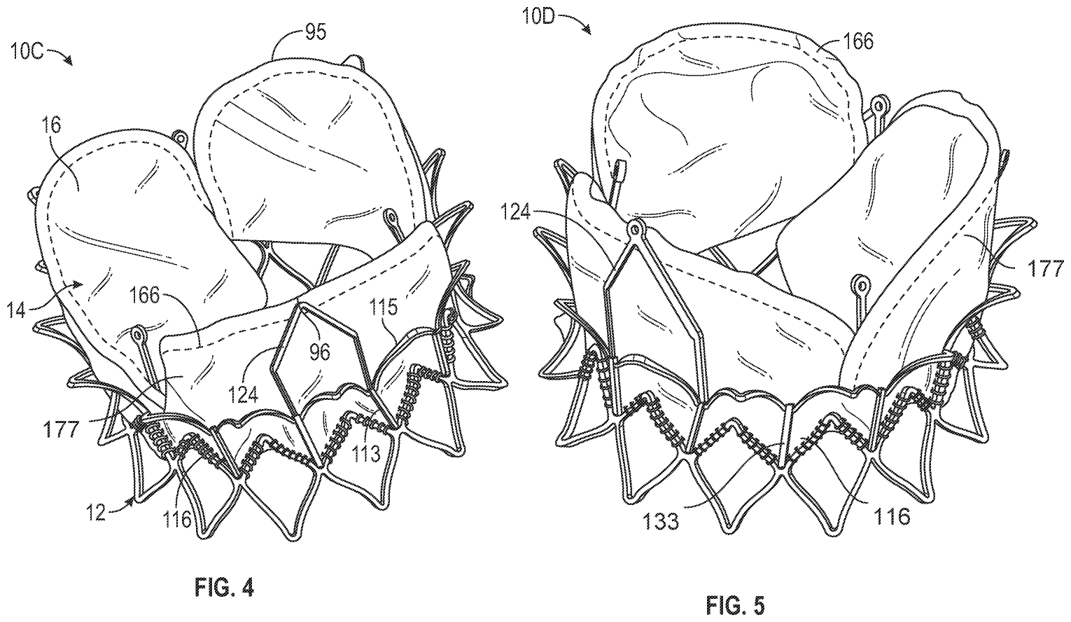

FIG. 3 shows a representative example of the valve prosthesis 10 in an unexpanded configuration coupled to an anchor 15. In some embodiments, the anchor 15 may comprise a spiral shape that, for example, spirals around the valve prosthesis 10 in the unexpanded and/or expanded configuration. In other embodiments, the anchor 15 can comprise a flat spiral or anchor that resides in a singular plane (e.g., a plane orthogonal to a longitudinal axis of the valve prosthesis). The anchor 15 can have a free end 22. In some cases, the free end 22 of anchor 15 can be useful during deployment of the anchor 15 in a native heart valve (e.g., by ensnaring chordae or other structures when the prosthesis 10, anchor 15, and/or delivery device are rotated around longitudinal axis of the valve prosthesis 10). The anchor 15 may be directly coupled to the frame structure 12, for example at a first end (e.g., a proximal end) or a second end (e.g., a distal end) thereof. Alternatively, the anchor 15 can be physically uncoupled from the frame structure 12 while providing an anchor for the frame 12 as the frame expands within the native valve orifice (thereby sandwiching tissue between the frame 12 and the anchor 15). In some embodiments, the frame structure 12 can be at least partially held in place within the native valve via interaction with the anchor 15. For example, the expanded diameter of the frame structure 12 can be greater than or equal to the inner diameter of the spiraled anchor 15 such that the frame structure 12 expands into and engages with the anchor 15 (with native valve leaflets, chordae, or other tissue therebetween).

A longitudinal axis of the anchor 15 may be co-axial or concentric with a longitudinal axis of the delivery device when the anchor 15 is in the deployed configuration. In some embodiments, the deployed anchor 15 may be detachably coupled to a delivery device prior to deployment of the valve prosthesis 10. For example, the anchor 15 can be deployed from a delivery device and held with a tether until the frame structure 12 is expanded within the native valve orifice and the anchor 15.

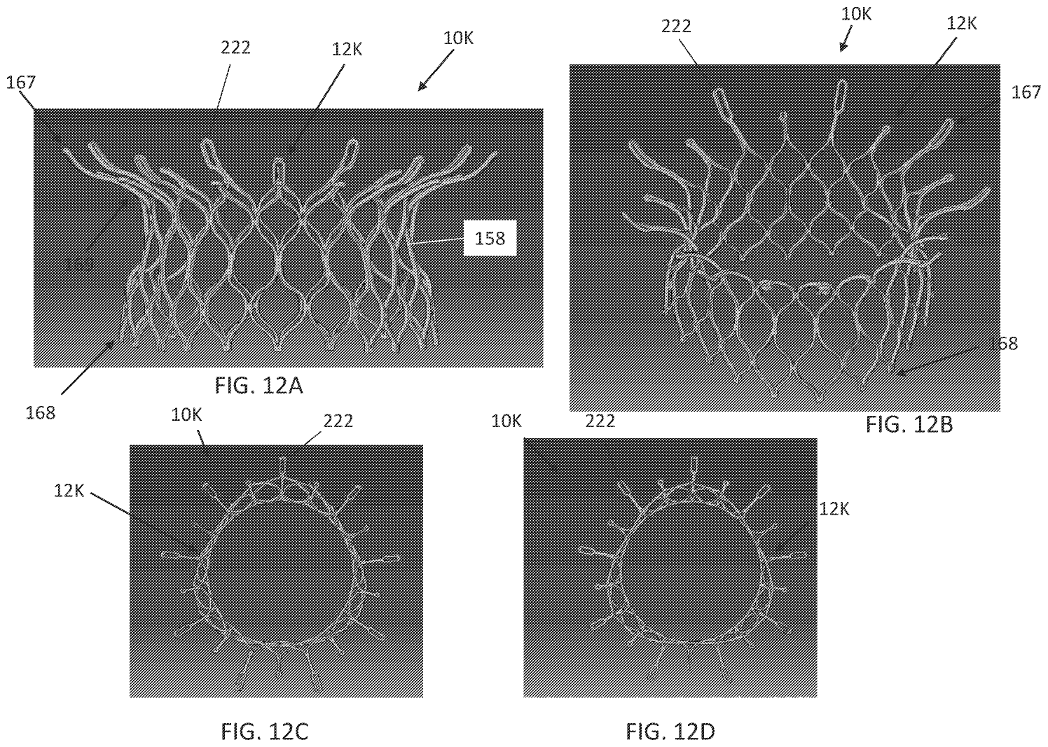

In some embodiments, the valve prostheses 10 described herein can include one or more flared portions to engage with the anchor 15 and/or help prevent the valve prostheses 10 from sliding through a valve orifice. For example, as shown in FIG. 10, the frame structure 12 of valve prosthesis 10I can include an atrial flared portion 157 extending radially outwards from a central annular portion 158. The atrial flared portion 157 can, for example, extend into the atrium of the heart from the central annular portion 158 when valve prosthesis 10 is deployed in a native mitral valve. Alternatively, or in combination, the atrial flared portion 157 can contact a tissue of the atrium of the heart, e.g., a mitral valve annulus when valve prosthesis 10 is deployed in a native mitral valve.

The valve prostheses 10 described herein may comprise a first and second opposite ends, the first end (e.g., the proximal end) oriented nearest the atrium when the valve prosthesis 10 is deployed in the orifice of a native mitral valve and the second end (e.g., the distal end) oriented nearest the ventricle when the valve prosthesis 10 is deployed in the orifice of a native mitral valve. Alternatively, the frame structure 12 may be configured to sit entirely below the native valve when the frame structure 12 is anchored to the native valve. In some cases, a first portion of frame structure 12 can be disposed in a longitudinal location nearer to a first end of the valve prosthesis 10 than the second portion of frame structure 12 (e.g., when the frame structure is in an unexpanded configuration). A first portion and/or second portion of frame structure 12 can have a first longitudinal end and a second longitudinal end. In some cases, a first longitudinal end of frame structure 12 can be oriented nearer to a first end of valve prosthesis 10 than a second longitudinal end of frame structure 12. In some cases, a second longitudinal end of frame structure 12 is oriented nearer to a second end of valve prosthesis 10 than a first longitudinal end of frame structure.

Any of the frame structures 12 described herein can provide structural strength to valve prosthesis device 10. For example, the frame structure 12 can be used to anchor the valve prosthesis 10 in position at a target location of a subject (e.g., in the orifice of a heart valve, such as a mitral valve or tricuspid valve).

The valve prostheses 10 described herein may include one or more valve segments 14 disposed therein to replace the native valve leaflets. For example, the valve segment 14 can include a plurality of leaflets 16, e.g., that form a biocompatible one-way valve. Flow in one direction may cause the leaflets 16 to deflect open and flow in the opposite direction may cause the leaflets 16 to close.

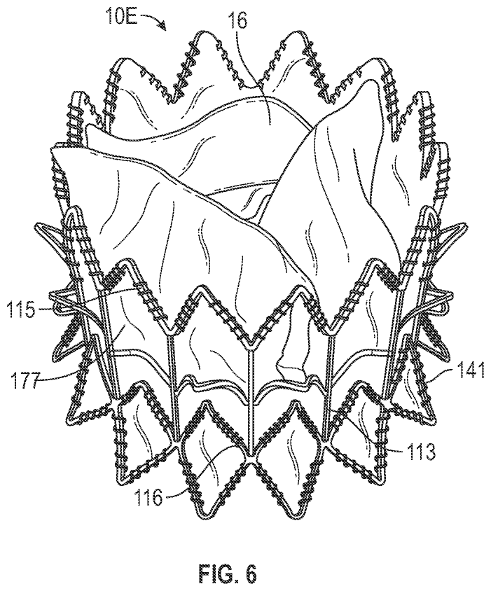

Any of the valve segments 14 described herein may be formed of multi-layered materials for preferential function. Referring to FIG. 4, for example, the valve prosthesis 10C may include a valve segment 14 having a seal 177 (also called an outer leaflet, outer layer, or skirt) positioned radially between leaflets 16 (also called inner leaflets or the inner layer) and the frame structure 12. The seal 177 can be a single piece wrapped around the leaflets 16 or can be individual pieces shaped to match the leaflets 16. In some cases, the seal 177 and/or leaflets 16 can be formed from or coated with a material to confer an advantage upon the valve segment 14. For example, a layer or surface of a valve segment 14 can be formed from or coated with a biocompatible material. In some cases, a layer or surface of a valve segment 14 can be formed from or coated with an anti-thrombotic material. In some cases, a valve segment 14 (or portion thereof, such as a leaflet 16 of the valve segment) comprises a synthetic material. In some cases, a valve segment 14 (or portion thereof, such as a leaflet) comprises a biological tissue. In many cases, a valve segment 14 (or portion thereof, such as a leaflet) comprises pericardial tissue. In some embodiments, a valve segment 14 (or portion thereof, such as a leaflet 16 of the valve segment 14) comprises a decellularized biological tissue. For example, a valve segment 14 (or portion thereof, such as a leaflet 16 of the valve segment) can include decellularized pericardium.

The valve segment 14 may be attached to a frame structure 12, which can in turn be attached to the anchor 15. The frame structure 12 may be connected to the anchor 15 before or after the frame structure 12 has been deployed adjacent a native valve. The frame structure 12 may be attached to the valve segment 12, for example, via attachment of the frame structure 12 to the seal 177, which can in turn be attached to the leaflets 16.

In some embodiments, two or more portions of a valve segment 15 (e.g., two or more leaflets 16, and/or seal 177) can comprise a single piece of material (e.g., a single piece of biological or synthetic tissue formed into the shape of a functional valve). In some cases, two or more portions of a valve segment (e.g., two or more of a first and second leaflet 16, and/or the seal 177) can be joined together. In some embodiments, two or more portions of a valve segment (e.g., two or more of a first and second leaflet 16, and/or the seal 177) can be joined together by suturing the two or more portions together (e.g., at sutured coupling 166 shown in FIG. 4). In some cases, 1, 2, 3, 4, 5, or more than 5 leaflets 16 can be coupled to a single seal 177.

In many cases, leaflet coupling 166 is disposed at an inflow end of valve prosthesis 10 (i.e., closest to the source of flow through the device, e.g., caused by a contracting heart chamber) when deployed. In some cases, coupling two or more portions of a valve segment 14 at the inflow end of valve prosthesis 10 (or portion thereof) allows the valve segment 14 to fold or collapse (e.g., radially away from a longitudinal axis of valve prosthesis device 10) during contraction of a heart chamber upstream of the deployed device (i.e., during diastole). Further, in some cases, coupling two or more portions of a valve segment 14 at the inflow end of valve prosthesis 10 causes the valve segment 14 to expand (e.g., radially toward a longitudinal axis of valve prosthesis device 10) during refilling of a heart chamber upstream of the deployed device (i.e., during systole). This expansion of the valve segment 14 can, for example, result in billowing or parachuting of the valve segment 14 (e.g., between the seal 177 and the leaflets 16) to block the flow of blood therethrough.

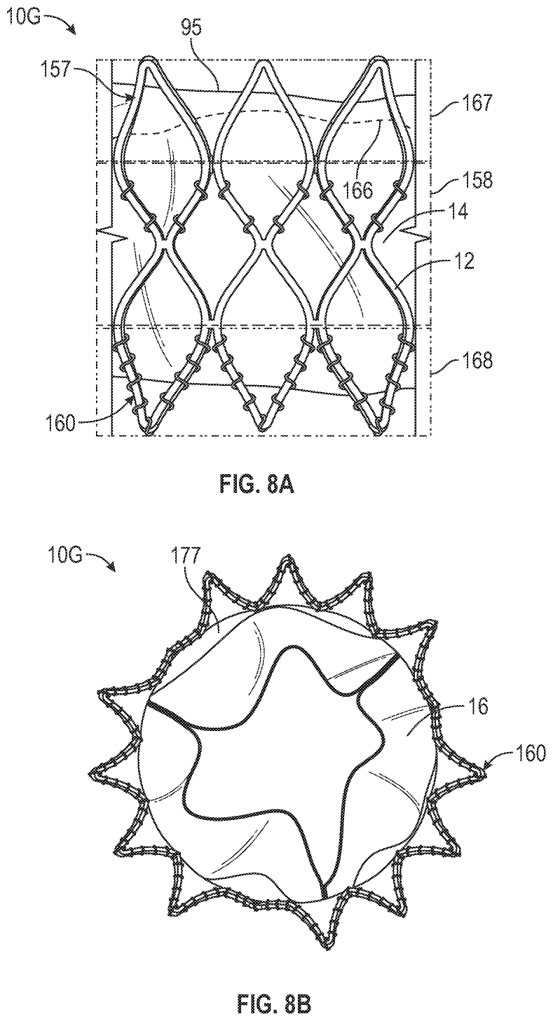

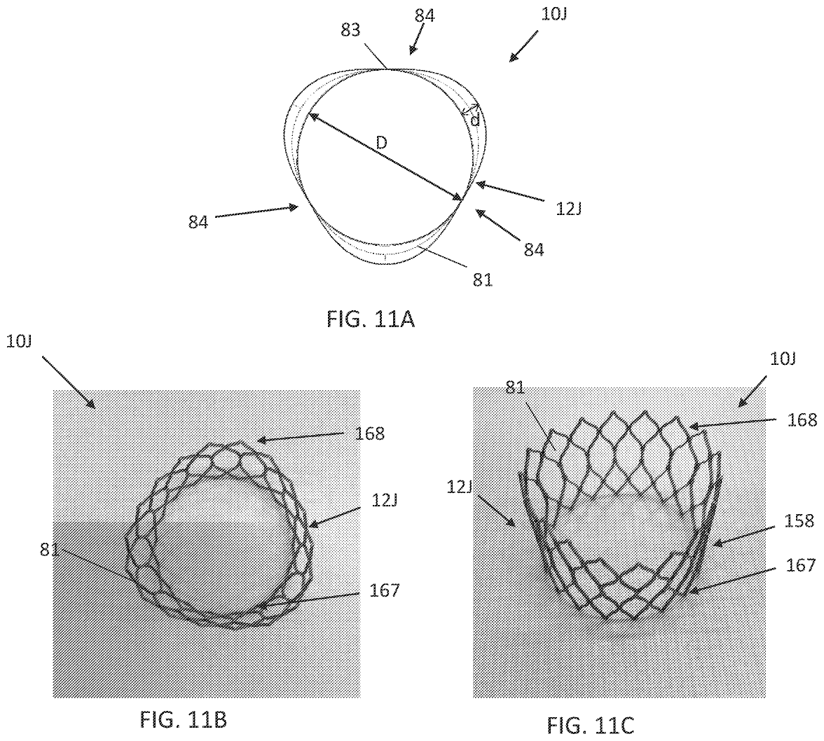

As shown in to FIG. 4, the valve segment 14 can be attached to one or more struts 113 of the frame structure 12. In some embodiments, a portion of a valve segment 14 (e.g., leaflets 16 or seal 177) can be sutured to the central annular portion 158 of frame structure 12 and not to the inflow portion of frame structure 12 or the outflow portion of frame structure 12 (e.g., can be unattached to the distal arches 116 and the proximal arches 115 as shown in FIG. 4). In some embodiments, a portion of a valve segment 14 (e.g., leaflets 16 or seal 177) can be sutured to one or more outflow portion of frame structure 12 and not to the inflow portion of frame structure 12 (e.g., can be sutured to one or more distal arches 116 but not one or more proximal arches 115 as shown in FIG. 9A). In some embodiments, a portion of a valve segment 14 (e.g., leaflets 16 or seal 177) can be sutured to one or more outflow portion of frame structure 12 and to the inflow portion of frame structure 12 (e.g., can be sutured to one or more distal arches 116 and also to one or more proximal arches 115 as shown in valve prosthesis 10E of FIG. 6). In some embodiments, an inflow end of the valve segment 14 can be substantially unsupported by the frame 12 while the outflow end of the valve segment 14 can be fully supported by and within the valve segment 14 (as shown in FIG. 4). The valve segment 14 (or portion thereof, such as the seal 177) can be coupled to the frame 12 continuously around the inner circumference of the frame 12 (e.g., at a distal or outflow end of valve prosthesis device 10).

In some cases, the amount of attachment of a valve segment 14 (e.g., a valve leaflet 16) to the frame structure 12 can be minimized, which can advantageously enhance ease of delivery and reduce the required length of the frame, thereby reducing the chance of thrombosis and reducing the chance of blocking the outflow from the ventricle to the aorta. Minimizing the frame structure 12 can also improve the speed and cost of fabrication of the valve prosthesis device 10.

In some embodiments, a leaflet 16 that is attached to a first portion of frame structure 12 (e.g., one or more struts 113) at a distal end of frame structure 12 can be unattached at a proximal end of the frame structure 12 (e.g., a strut or portion thereof at a proximal end of frame structure 12). In some cases, valve prosthesis devices 10 in which a valve segment 14 is attached at a proximal end of frame structure 12 and is unattached at a proximal end of frame structure 12 (and/or at a proximal end of valve segment 14) may require less metal and/or fewer struts than a valve prosthesis 10 in which a valve segment 14 is attached at both a proximal end and a distal end of the frame structure 12 of the valve prosthesis device 10. In some cases, minimizing the amount of metal used in the structure of valve prosthesis 10 (e.g., by reducing the number and/or length of struts in valve prosthesis device 10) can reduce the risk of thrombus formation and can improve the ease with which the device is deployed at a target location.

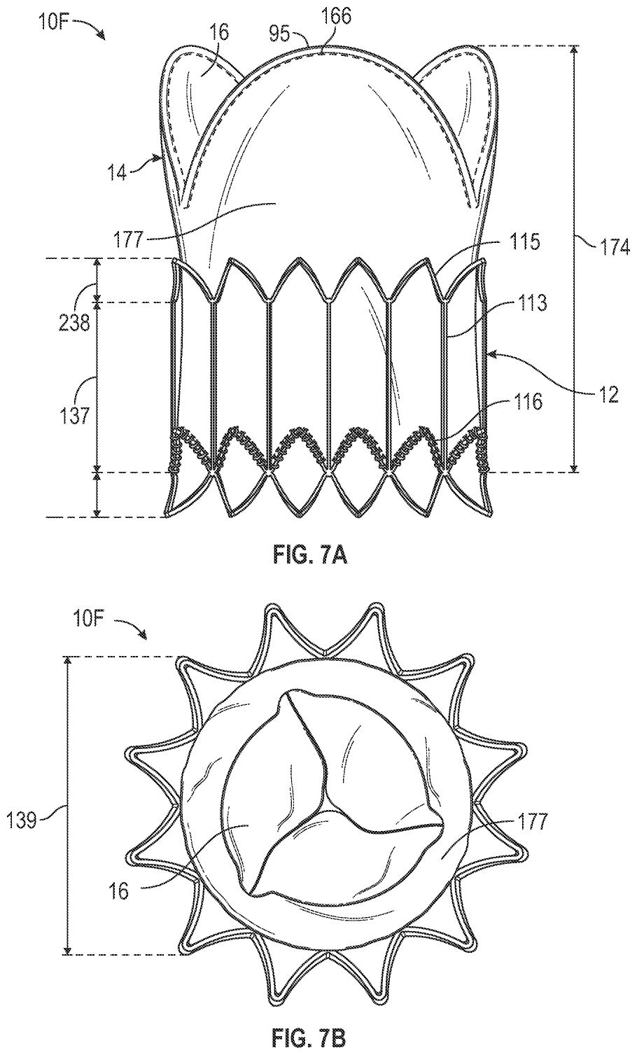

Further, the valve segment 14 can be configured to be substantially unsupported at the inflow edge 95 of the valve segment 14. For example, as shown in FIG. 4, the entire inflow edge 95 of valve segment can be unsupported with the exception of minimal valve supports 124 positioned at the nadir 96 of each leaflet 16. The valve supports 124 can have a pointed proximal tip and can extend, for example, from two neighboring struts 113 of the frame structure 12. The minimal valve supports 124 can help prevent the valve segment 14 (e.g., the seal) from collapsing radially inwards in the outflow direction (i.e., towards the ventricle) when implanted in the heart. FIG. 5 shows a valve prosthesis 10D that is similar to valve prosthesis 10C of FIG. 4 except that the valve support 124 of FIG. 5 includes an aperture 97 for suturing the leaflet 16 to the valve support 124.

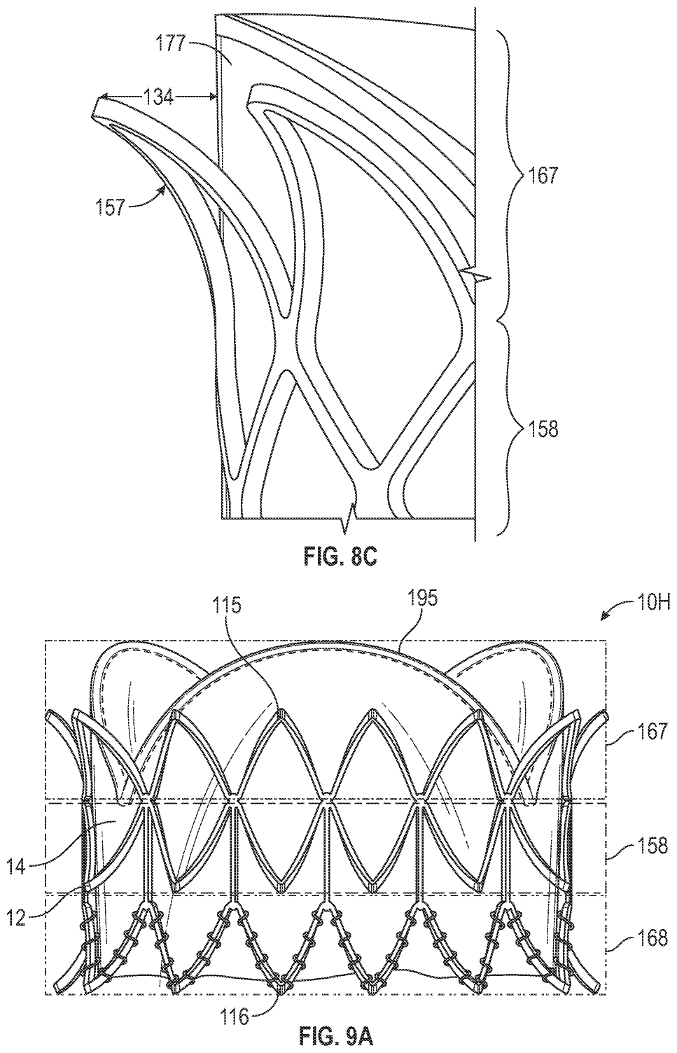

FIGS. 7A-7B show a valve prosthesis 10F wherein the inflow edge 95 of valve segment 14 is completely unsupported (i.e., does not include any valve supports thereto).

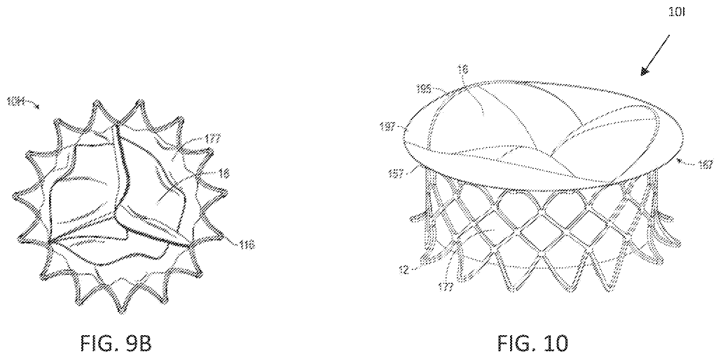

FIGS. 8A-8C show another valve prosthesis 10G wherein the inflow edge 95 of valve segment 14 is completely unsupported (i.e., does not include any valve supports thereto). Indeed, as shown in FIGS. 8A-8C, the prosthesis 10G can include an inflow portion 167, a central annular portion 158, and an outflow portion 168. The valve segment 14 can be fully circumferentially supported by the frame structure 12 within the central annular section 158. However, the valve segment 14 can be unsupported by and/or unconnected from the frame structure 12 in the inflow portion 167. Further, the frame structure 12 can flare radially outwards within the inflow portion 167. The flared portion 157 of the frame structure 12 can include a plurality of discrete flanges (i.e., formed from flared proximal arches 115) and can, for example, serve to help engage with an external anchor. Moreover, due to the flared portion 157, the valve segment 14 can be radially spaced away from the frame structure 12 within the inflow portion 167 by a distance 134 (see FIG. 8C). In some embodiments, the distance 134 can be 1-10 mm, such as 2-8 mm, such as 3-5 mm. Finally, the frame structure 12 can also flare radially outwards within the outflow portion 168. The flared portion 160 of the frame structure 12 can also serve to help engage with an external anchor 15. For example, the external anchor 15 can sit between the flared portions 157, 160 upon implantation.

FIGS. 9A-9B show another valve prosthesis 10H that is similar to valve prosthesis 10G of FIGS. 8A-8C except that substantially all of the inflow edge 195 extends proximally beyond the proximal arches 115 of the frame structure 12. When the leaflets are closed (as shown in FIG. 9B), the fluid pressure can act to fill the space created by the leaflets 16 and the seal 177, thereby preventing inward motion or collapse of the valve segment 14.

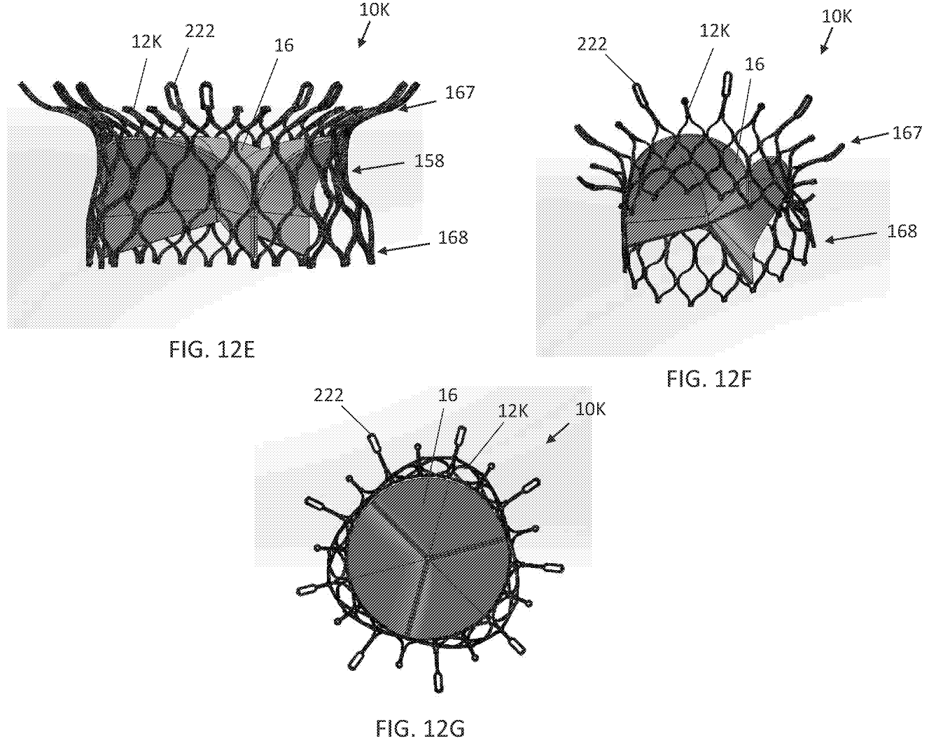

FIGS. 11A-11C show another valve prosthesis 10J that includes an inflow portion 167, a central portion 158, and an outflow portion 168. The valve 10J is similar to the prostheses 10G and 10H described herein except that the outflow portion 168 of the frame 12J protrudes outwards relative to the frame central portion 158 at three protruding sections 81. In some embodiments, the protruding sections 81 can create a generally triangular cross-section. The triangular cross-section can be referred to as a convex triangular cross-section (i.e., a triangle wherein an angle along an interior surface of the triangular cross-section is 180 degrees or less along an entire inner perimeter). While referred to herein as a “triangle,” it will be appreciated that the frame structures described herein include generally triangular shapes that have rounded corners (i.e., where the protruding sections 81 form the rounded corners). In some embodiments, the convex triangular cross-section can be an equilateral triangle. A central point 83 (e.g., midpoint) of each of the sides 84 of the convex triangle can be substantially radially aligned with both the central portion 158 and with the nominal valve inner circumference (indicated by diameter D). The central point 83 can also be the location of the commissure attachment mechanisms that are configured to attach the frame to the commissures of the valve leaflets. The nominal valve inner circumference D can further define a cylindrical blood lumen or blood flow path through the valve frame. As shown, the sides 84 of the convex triangle are non-inverting (i.e., the sides do not bend, buckle, or invert inwards towards the center of the valve frame). The sides 84 can have a slight radius of curvature, as shown, but that radius of curvature can approach or reach zero at the central point 83 of each side 84. Although referred to herein as a convex triangular cross-section, the triangular cross-section of the outflow portion 168 can additionally be referred to as a rounded triangle, a triangle with rounded corners, a triangle without inversions, or a trircle. Similarly, the cross-sectional shape can more generally be referred to as a convex polygon, a polygon without inversions, or a polygon with rounded corners. In some embodiments, the shape of the outflow portion 168 can be defined by polar cosine geometry as follows:

$$x = ( - \frac{( {R}_{Max} - {R}_{Min} )}{2} cos ( 3 \text{?} ) + \frac{{R}_{Max} + {R}_{Min}}{2} ) cos ( t )$$

$$y = ( - \frac{( {R}_{Max} - {R}_{Min} )}{2} cos ( 3 \text{?} ) + \frac{{R}_{Max} + {R}_{Min}}{2} ) sin ( t )$$

$$Where : 0 \leq t \leq 2 \text{?}$$

$$\text{?} \text{indicates text missing or illegible when filed}$$

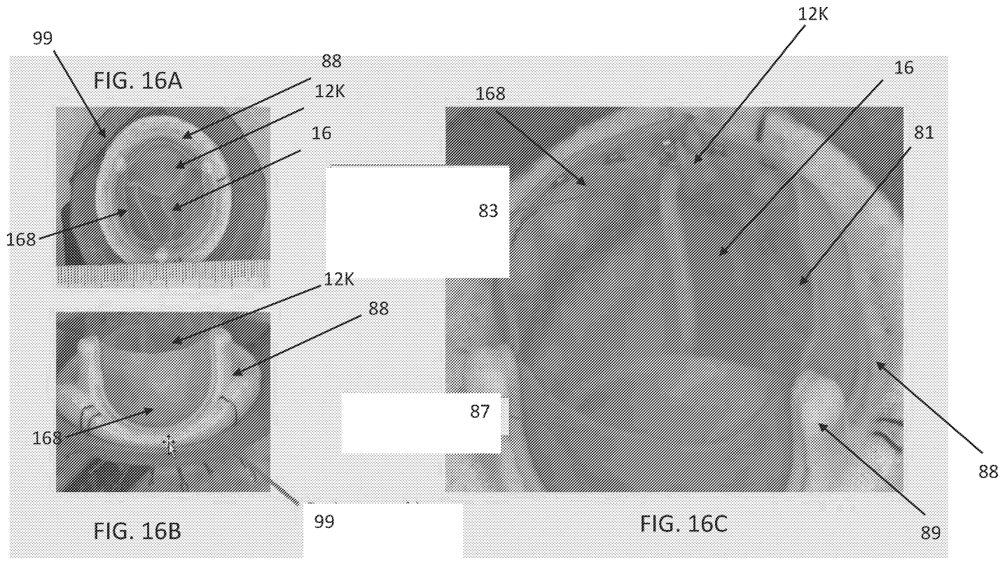

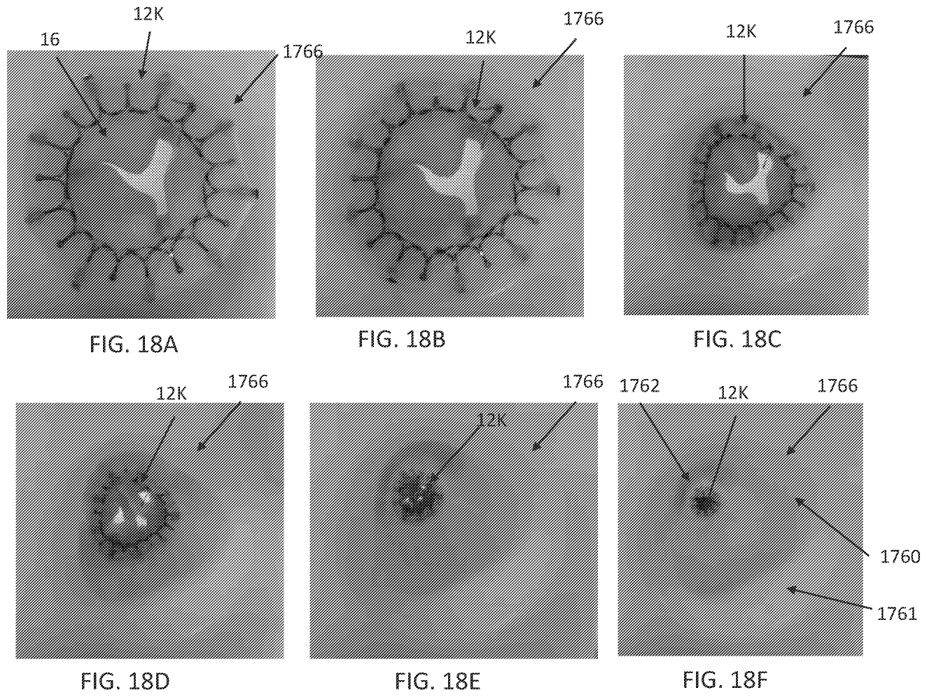

FIG. 15 shows a graph of curvature v. angle around the circumference of the outflow portion 168. As indicated on the graph, the outflow portion 168 can have a positive (i.e., convex) curvature around the entire circumference thereof with an increase in curvature at each of the three protruding sections 81. The sections of the graph that reach or approach zero can correspond to central points 83 along the sides of the convex triangle. As described above, these central points can be the location of the commissure attachment mechanisms of the frame that are configured to attach to the commissures of the leaflets. The graph shown in FIG. 15 can correspond to any position along the axial length of the outflow portion 168 (i.e., the curvature of the outflow portion 168 can remain at or above zero along its entire length). Advantageously, maintaining the curvature at or above zero can help ensure that the outflow portion 168 does not invert during sheathing, but rather collapses from the self-expanded convex triangular cross-section down to a substantially circular cross-section (e.g., as shown with respect to FIGS. 18A-18F, described further below).

Leaflets 16 (as described elsewhere herein) can be configured to be attached to the frame 10J, such as with commissure attachment mechanisms of the frame. Having an outflow portion 168 with a curvature at or above zero can additionally advantageously help maintain stability of the frame 12J as the leaflets 16 open and close. The leaflets 16 can be attached along the central points 83 of the outflow portion 168. The outward projection of the protruding sections 81 (relative to the nominal diameter D of the frame 10J) can advantageously ensure that the outflow edges of the leaflets 16 are held away from the inner circumference of the frame structure 12J during both opening and closing of the leaflets 16. For example, the outflow edges of the leaflets 16 can be held a distance d away from the interior circumference of the frame 12J at the protruding sections 81, such as at least 1.5 mm-4 mm away, such as 2-3 mm, such as approximately 2.5 mm, during both opening and closing of the leaflets 16. Because the outflow edges of the leaflets 16 are held away from the inner circumference of the frame 12J, deformation to the leaflets 16 can be minimized even when the frame structure 12J is deformed (e.g., upon implantation). Additionally, the spacing between the leaflets 16 and the inner circumference of the frame structure 10J at the protruding sections 81 can advantageously enable more blood to flow through that area while the leaflets 16 are opening to help prevent blood stagnation between the frame 10J and the leaflets 16. Finally, the spacing between the leaflets 16 and the inner circumference of the frame 10J can help maintain durability of the leaflets 16 (i.e., can prevent frictional wear of the leaflets 16 over time).

Additionally, as a result of their attachment to the frame 12J at the central points 83, the leaflets 16 can extend substantially straight down (i.e., towards the outflow end) from the central annular portion 158 (i.e., without curving radially outwards). That is, the nominal valve inner diameter formed by the central points 83 can be approximately equivalent to the inner diameter D of the frame 12J at the central annular portion 158 (and/or at the inflow portion 167). Thus, the leaflets 16 can form a substantially cylindrical flow path for blood passing therethrough.

The inflow portion 167 and the central portion 158 of the frame 12J can be cylindrical (as shown) or flared as described elsewhere herein. The inflow portion 167 can extend into the atrium of the heart while the convex triangular outflow portion 168 can extend into the ventricle.

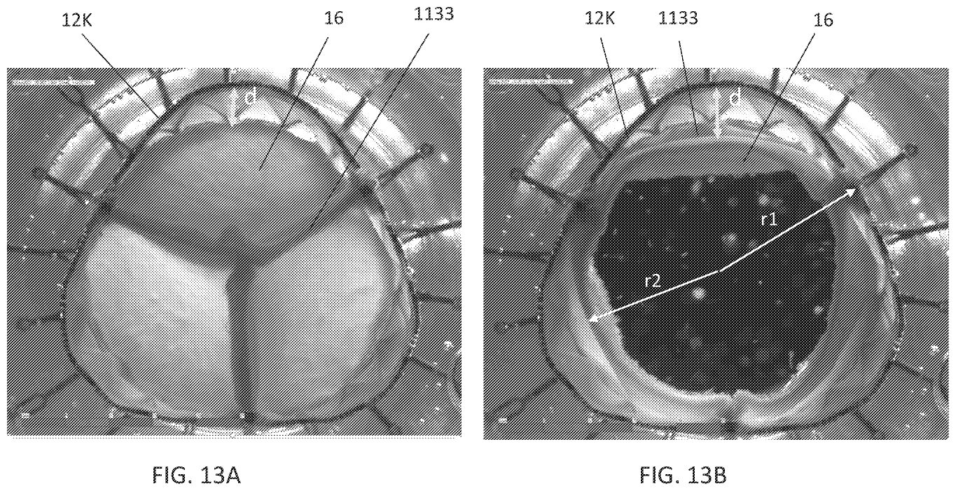

FIGS. 12A-12G show another exemplary valve prosthesis 10K. Valve prosthesis 10K is similar to valve prosthesis 10J except that the inflow portion 167 is flared radially outwards and includes a plurality of paddles 222. As shown in FIG. 12A, the central portion 158 of the frame can be narrower (e.g., have a smaller diameter) than both the inflow portion 167 and the outflow portion 168. The diameter of the central portion 158 can define the blood flow lumen or conduit through the frame. Also as shown in FIG. 12A, the inflow portion 167 is flared radially outwards to form a shelf or ridge 169 in the inflow portion. In some embodiments, this shelf or ridge 169 can be configured to seat or engage the valve frame against the anatomy to help keep it in place. While the inflow portion generally flares radially outward with respect to the central portion of the frame, in some embodiments, as shown, the very proximal ends of the inflow portion can start to flare back towards the longitudinal axis of the frame, as shown, particularly the section proximal to the shelf or ridge 169. As with the prosthesis 10J described above, prosthesis 10K can include an outflow portion 168 of the frame that protrudes outwards relative to the frame central portion 158 at three protruding sections. In some embodiments, the protruding sections can create a triangular cross-section. The triangular cross-section can be referred to as a convex triangular cross-section. The convex triangular cross section can have rounded corners (i.e., where the protruding sections form the rounded corners). In some embodiments, the convex triangular cross-section can be an equilateral triangle.

The axial spacing at the central annular portion 158 between the flared inflow portion 167 and the protruding sections 81 of the outflow portion 168 can enable the anchor 15 (described elsewhere herein) to lodge therebetween to better anchor the prosthesis 10K in place. The paddles 222 can be configured to extend from the flared inflow portion 167. The paddles 222 can be configured to remain above (or proud of) other portions of the flared inflow portion 167 when implanted. The paddles 222 can advantageously enable grasping and/or manipulation of the valve frame 12K during deployment. Additionally, at least a portion of the paddles 222 (e.g., the tips of the paddles 222) can be straight (i.e., not formed into cells) so as to be non-foreshortening during deployment. The non-foreshortening portions can advantageously help with trackability and sheathing during delivery of the frame. The non-foreshortening portions can additionally advantageously provide increased apposition on the atrial side, allowing for a reduction in length of the forsehortening elements (e.g., the diamond-shaped cells), and thus increasing radial stiffness in the forsehortening section of the valve frame 12K. Further, in some embodiments, the extended paddles 222 can include at least two inflection points (e.g., at the non-foreshortening portions), which can help orient the tips of the flared inflow portion 167 away from the anatomy, thereby making the tips less traumatic.

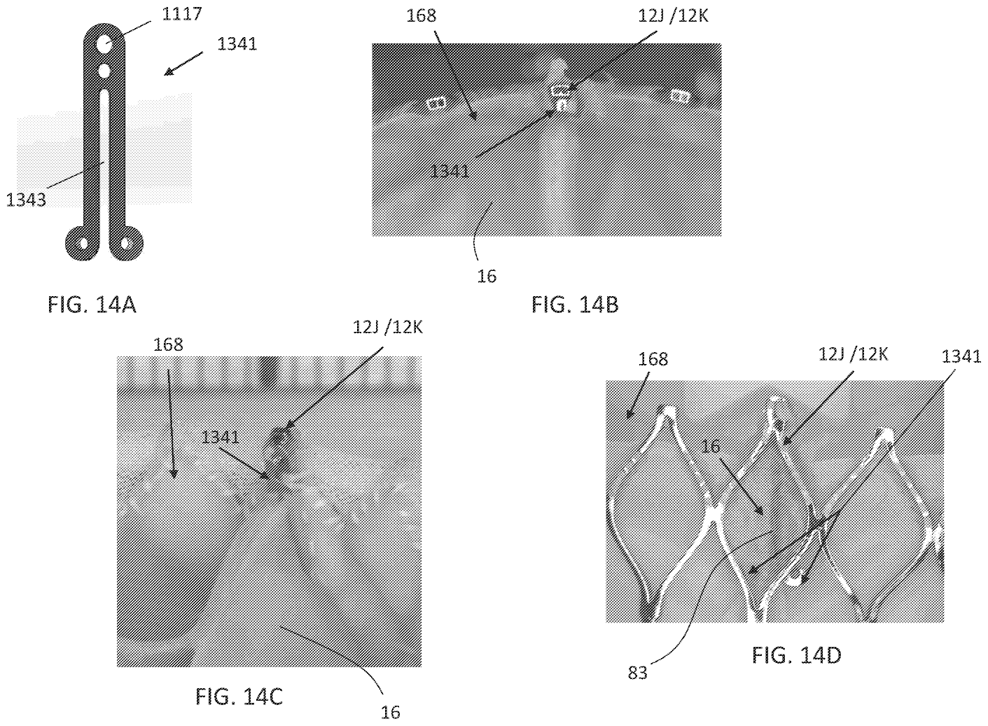

FIGS. 13A-13B show the closed (FIG. 13A) and open (FIG. 13B) positions of leaflets 16 relative to a frame 12K upon implantation (e.g. in the mitral valve orifice). As shown, the edges 1133 of the leaflets 16 remain a distance d away from the frame 12K both while the leaflets 16 are fully opened and fully closed. As shown, the distance d from which the leaflets remain away from the frame 12K corresponds to the protruding sections of the outflow portion of the frame relative to the central portion. In this embodiment, the protruding portions of the convex triangular cross-section do not include sections of the leaflets 16. Described another way, an outer portion of the leaflets 16 generally comprise a circular cross-section when fully opened and closed, and the protruding sections which form the convex triangular cross-section of the outflow portion generally do not include any leaflets disposed therein, providing the distance d between the leaflets and the frame 12K as shown. Also as shown in FIG. 13B, r1 is the radius from a central point in the frame to a commissure attachment mechanism of the frame, and r2 is the radius to from the central point to the (free) edges of a leaflet when the leaflets are fully open. In some embodiments, the radius r2 (to the leaflets) is at least as large as the radius r1 without contacting frame. This is facilitated by the protruding sections of the outflow portion of the frame, which allows the leaflets to open wider than the narrowest sections of the outflow region.

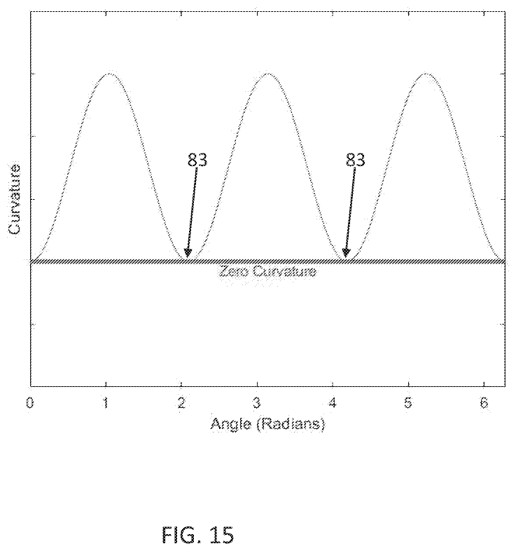

An exemplary attachment mechanism for attaching the leaflets 16 to the frame 12J or 10K at central points 83 is shown in FIGS. 14A-14D. The attachment mechanism can include a commissure attachment pin 1341 configured to extend along the central points 83 at the outflow portion 168. The attachment pin 1341 can include an axial slot 1343 therein configured to allow passage and/or wrapping of tabs of the leaflets 16 therethrough. One or more sutures can attach the pin 1341 (via suture holes 1117) to the frame 12J/12K, thereby also attaching the leaflets 16 to the frame 12J/12K. Advantageously, the attachment mechanism for the leaflets 16 can be relatively thin and flush with the frame 10J/10K, thereby simplifying collapsing and/or sheathing of the frame 10J/10K.

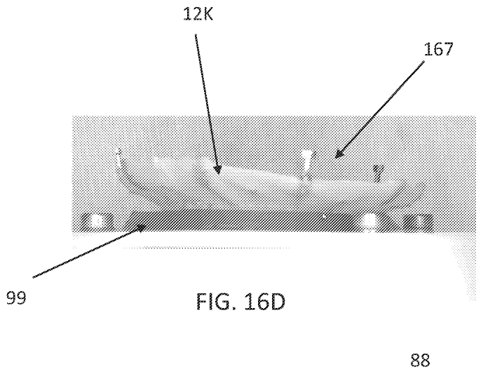

Referring to FIGS. 16A-16D, in some embodiments, a frame 12K (or 12J) with leaflets 16 can be configured to be used as a valve-in-valve (e.g., can be placed inside of a surgical valve 88, such as a stentless surgical valve, that is already implanted within the annulus 99). The convex triangular shape of the outflow portion 168 can advantageously ensure a stable and sealing fit inside of the surgical valve 88. That is, as shown best in FIG. 16C, the central points 83 (and, correspondingly, the commissure attachment mechanisms) of the frame 12K can align with the commissure posts 89 of the surgical valve 88. The protruding sections 81 can then push out against the leaflets 87 of the surgical valve 88, creating a tight seal. Additionally, as shown in FIG. 16D, the flared inflow portion 167 may radially overlap with the surgical valve 88 (e.g., extend radially beyond the surgical valve 88), thereby providing additional sealing.

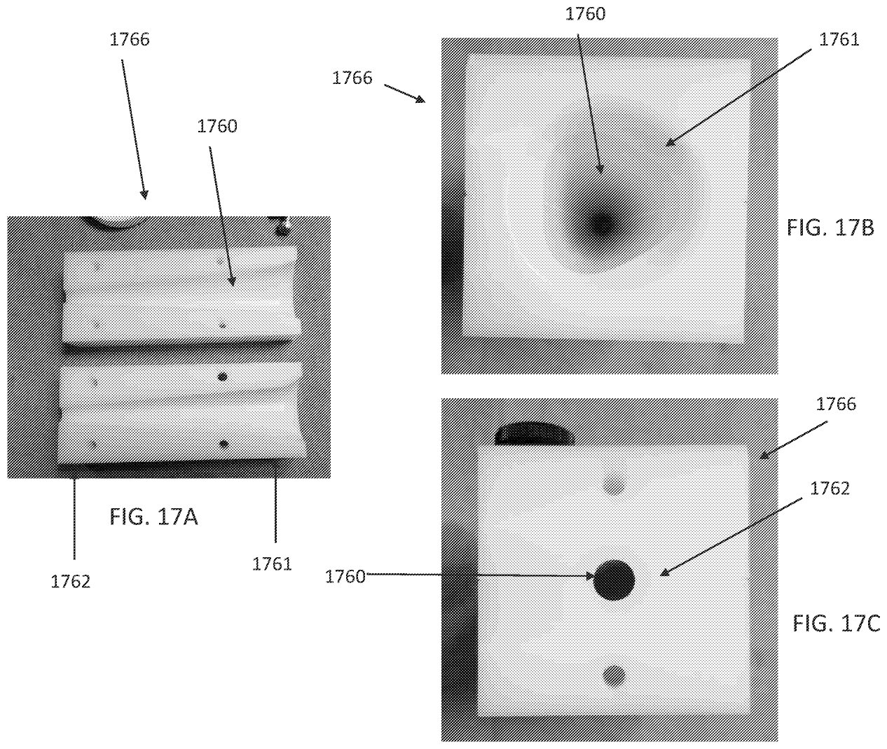

An exemplary loading tool 1766 for loading the frame 12J or 12K into a sheath is shown in FIGS. 17A-17C. The loading tool 1766 can include a central opening 1760 with an input end 1761 having a shape similar to the outer contours of the frame 10J or 10K (e.g., with a convex triangular cross-section) and an output end 1762 having a circular cross-section. Further, the central opening 1760 can decrease in diameter from the input end 1761 to the output end 1762. FIGS. 18A-18F show a frame 12K (and corresponding leaflets 16) as it is pulled from the input end 1761 to the output end 1762 of the loading tool 1766. A delivery device (not shown) can be, for example, positioned at the output end 1762 to facilitate sheathing of the frame into the delivery device. At FIG. 18A, the frame 12K in the expanded configuration fits within the input end 1761 such that the convex triangular outflow portion abuts the convex triangular walls of the central opening 1760 and the flared inflow portion is positioned external to the central opening 1760. At FIGS. 18B-18C, the entire frame 12K takes on a reduced profile convex triangular cross-section as the frame 12K is pulled into the central opening 1760 towards the output end 1762. At FIGS. 18D-18F, the frame 12K begins to reduce in size to a substantially circular cross-section so as to fit within a sheath. Advantageously, the frame 12K can reduce in profile without inverting during sheathing, thereby ensuring the stability of the frame 12K upon implantation.

In some embodiments, the struts of the frames 12J or 12K (e.g., of the convex triangular portions of the frame 12J or 12K) can be varied in thickness or length to help ensure uniform expansion of the frame 12J.

It should be understood that the entire frame structures 12J and 12K and/or features of the frame structures 12J and 12K can be interchanged and/or combined with any of the frame structures 12 described herein.

In some embodiments described herein, the inflow edge 95 of the leaflets can be entirely unsupported except at commissures of the leaflets 16, such as except for at the central points 83 and/or the minimal valve supports 124.

Referring to FIGS. 7A-7B, in some cases, the size of a valve prosthesis 10F (which can correspond to any of the valve prostheses 10 described herein), e.g., the magnitude of a frame height 137 of a valve prosthesis 10F in an expanded configuration) can be measured relative to one or more structures of the valve prosthesis 10F (e.g., a valve segment height of the valve prosthesis device in an expanded configuration, a leaflet height 174 when the device is expanded, and/or a diameter 139 of an expanded frame body) and/or relative to one or more biological structures (e.g., the mean diameter of a heart valve in which the device is deployed).

In some cases, the height 137 of a frame of the valve prosthesis 10F can be measured relative to the height 174 of a valve segment 14 of the valve prosthesis device 10F (e.g., valve segment height-to-frame height ratio, or VSTF ratio, e.g., a ratio of height 137 to height 174). In some cases, the height 174 of a valve segment 14 (or portion thereof, such as a valve leaflet) of an expanded valve prosthesis 10F is greater than the height of the frame of the valve prosthesis device (e.g., a VSTF ratio greater than 1).

A portion of frame structure 12, such as strut 113 and/or minimal valve support 124 (e.g., hoop structure) that can be used to provide frame structure 12 with compressive strength and/or resiliency can be made of a metal or a metal alloy. Representative examples of metals and metal alloys that can be used to form all or part of a portion of frame structure 12 include nickel-titanium alloys (NiTi), cobalt-chrome alloys, and stainless steel. A portion of a frame structure (e.g., strut 113 or minimal valve support 124) can be made of a material comprising one or more of the following metals: titanium, aluminum, cobalt, chrome, molybdenum, vanadium, zirconium, zinc, nickel, niobium, tantalum, magnesium, and iron. Specific titanium alloys that can be used include Ti-3Al-2.5V, Ti-5Al-2.5Fe, Ti-6-Al-4V, Ti-6Al-4V ELI, Ti-6Al-7Nb, Ti-15Mo, Ti-13Nb-13Zr, Ti-12Mo-6Zr-2Fe, Ti-45Nb, Ti-35Nb-7Zr-5Ta, and Ti-55.8Ni. A portion of a frame structure 12 can comprise a nickel-titanium alloy having equal or nearly equal amounts of nickel and titanium. For example, a nickel-titanium alloy can be 50 mol %, from 49.5 mol % to 50.5 mol %, from 49 mol % to 51 mol %, from 48.5 mol % to 51.5 mol %, from 48 mol % to 52 mol %, 47.5 mol % to 52.5 mol %, or from 47 mol % to 53 mol % nickel.

In some cases, a portion of valve prosthesis 10 can comprise a ceramic. For example one or more portions of frame structure 12 can comprise one or more of alumina, zirconia, quartz, pyrolytic carbon (e.g., pyrolytic carbon coated graphite), or a calcium phosphate such as hydroxyapatite.

A portion of valve prosthesis 10 can comprise a polymer (e.g., a sterilizable polymer and/or biocompatible polymer). In some cases, a polymer can comprise one or more of polyethylene (e.g., polyethylene terephthalate (PET), polytetrafluoroethylene (PTFE)), a fluoropolymer, silicone, polystyrene, nylon, polyurethane, thermoplastic polyurethane (TPU), polysiloxane, polylactide (PLA), polyglycolide (PGA), poly(lactide-co-glycolide) (PLGA), polycaprolactone (PCL) such as poly(s-caprolactone), poly(methyl methacrylate), hyaluronan, polydioxanone, polyanhidride, or trimethylene carbonate. In some cases, a polymer of a valve prosthesis 10 or portion thereof can be a co-polymer (e.g., a block co-polymer). In some cases, a polymer can be cross-linked (e.g., using ultraviolet light) to increase strength and/or resiliency of a polymer.

Materials comprising valve prosthesis 10 or a portion thereof (e.g., frame structure 12, fabric covering 112, or strut 113) can be formed into solid structures or meshes. For example, fabric covering 112 can comprise one or more materials (e.g., polymers such as polyester or nylon) formed into a fabric or mesh.

In some cases, valve prosthesis 10 or a portion thereof (e.g., valve leaflet 16) can comprise a cell-based tissue. The use of a cell-based tissue as a material for valve prosthesis 10 or a portion thereof can offer various advantages, such as decreased thrombogenicity, improved integration of an implanted valve prosthesis 10 with surrounding native tissue, improved material properties of the device or portion thereof, and, in some cases, decreased immune response. For example, a valve prosthesis 10 (or portion thereof) comprising a cell-based tissue can exhibit mechanical properties closer to those of a healthy valve under static and/or dynamic mechanical loading. A cell-based tissue derived from a subject's own tissue (e.g., stem-cell derived tissues) or from an allogenic source comprising all or a portion of valve prosthesis 10 can decrease the likelihood of immunogenic response after implantation, in some cases. In some cases, one or more cells of a cell-based tissue useful in a valve prosthesis 10 can be autologous, allogeneic, or xenogeneic relative to a subject in which the prosthetic valve device is deployed. Representative examples of sources of one or more cells of a cell-based tissue useful in a valve prosthesis 10 are a human, a pig, or a cow. One or more distal (or ventricular) surfaces of leaflet 16 can be fabricated from, coated with, or treated with a biocompatible material.

As would be understood by a person of skill in the art, various embodiments of valve segments, valve anchors, and frame anchors, can offer advantages for the treatment or replacement of a native valve.

It should be understood that any feature described herein with respect to one embodiment can be substituted for or combined with any feature described with respect to another embodiment.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.10% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A device for treating a diseased native valve in a patient, the device comprising:
a frame structure comprising:
an inflow portion; and
an outflow portion comprising a plurality of protruding sections configured such that a cross-section of the outflow portion forms a generally convex triangular shape; and
a valve segment positioned radially within the frame structure, the valve segment comprising a plurality of leaflets attached to the frame structure.

2. The device of claim 1, wherein each of the leaflets is attached to the frame at a central attachment point along a respective side of the convex triangular cross-section.

3. The device of claim 2, wherein the leaflets are unsupported by the frame except at the central attachment points.

4. The device of claim 1, wherein there is a gap between an interior diameter of the outflow portion at the protruding sections and an outflow edge of the valve segment.

5. The device of claim 1, wherein the inflow portion is substantially cylindrical.

6. The device of claim 1, wherein the inflow portion is flared radially outwards.

7. The device of claim 1, wherein a cross-section of the inflow portion is substantially circular.

8. The device of claim 1, wherein the outflow portion has a curvature at or above zero along an entire axial length thereof.

9. The device of claim 1, wherein a nominal inner diameter of the outflow portion is approximately equal to an inner diameter of the inflow portion.

10. The device of claim 1, wherein the frame structure is configured to self-expand from a collapsed configuration to an expanded configuration, further wherein the outflow portion comprises the plurality of protruding sections when the frame structure is in the expanded configuration.

11. The device of claim 10, wherein the collapsed configuration is substantially cylindrical.

12. The device of claim 1, wherein the device is configured to be placed inside a stentless surgical valve.

13. The device of claim 1, further comprising a spiral anchor configured to be placed around the frame structure.

14. The device of claim 1, wherein the convex triangular shape comprises a triangle wherein an angle along an interior surface of the triangular shape is 180 degrees or less along an entire inner perimeter.

15. The device of claim 1, wherein the protruding sections comprise curvature that forms rounded corners of the convex triangular shape.

16. The device of claim 1, wherein the convex triangular shape is an equilateral triangle.

17. The device of claim 1, further comprising a central portion between the inflow portion and the outflow portion.

18. The device of claim 17, wherein the convex triangular shape comprises three sides, wherein a midpoint of each of the three sides is substantially radially aligned with both the central portion and with a nominal inner circumference of the frame structure.

19. The device of claim 17, wherein the plurality of protruding sections protrude outwards relative to the central portion.

20. A device for treating a diseased native valve in a patient, the device comprising:
a frame structure comprising:
a flared inflow portion;
a plurality of paddles extending from the flared inflow portion, the plurality of paddles being configured to enable grasping and/or manipulation of the frame structure during deployment;
a central portion;
an outflow portion that protrudes outwards relative to the central portion at a plurality of protruding sections configured such that a cross-section of the outflow portion forms a generally convex triangular shape; and
a valve segment positioned radially within the frame structure, the valve segment comprising a plurality of leaflets attached to the frame structure.

21. The device of claim 20, wherein at least a portion of the plurality of paddles is non-foreshortening during deployment.

22. The device of claim 21, wherein a tip of the plurality of paddles is straight.

23. The device of claim 20, wherein the plurality of paddles include at least two points of curvature inflection.

24. A valve replacement system, comprising:
a frame structure comprising:
an inflow portion; and
an outflow portion comprising a plurality of protruding sections configured such that a cross-section of the outflow portion forms a generally convex triangular shape; and
a valve segment positioned radially within the frame structure, the valve segment comprising a plurality of leaflets attached to the frame structure;
a loading tool including a central opening with an input end having a cross-section similar to the generally convex triangular shape of the outflow portion of the frame structure and an output end having a generally circular cross-section; and
a delivery device;
wherein the frame structure is configured to be passed through the loading tool to facilitate sheathing of the frame structure into the delivery device.

25. The system of claim 24, wherein the central opening decreases in diameter from the input end to the output end.

26. (canceled)
